(19) United States
(12) Patent Application Publication
(10) Pub. No.: US 2022/0328153 A1
(43) Pub. Date: Oct. 13, 2022

(54) METHOD, SYSTEM, AND PLATFORM FOR DELIVERY OF EDUCATIONAL INFORMATION AND PELVIC HEALTH MANAGEMENT

(71) Applicant: Renalis LLC, Cleveland, OH (US)

(72) Inventor: Missy Lavender, Fontana, WI (US)

(73) Assignee: Renalis LLC, Cleveland, OH (US)

(21) Appl. No.: 17/808,957

(22) Filed: Jun. 24, 2022

Related U.S. Application Data
(63) Continuation of application No. 17/454,483, filed on Nov. 10, 2021, now abandoned.
Application No. 17/454,483 is a continuation of application No. 17/336,294, filed on Jun. 1, 2021, now abandoned.
Application No. 17/336,294 is a continuation of application No. 16/780,892, filed on Feb. 3, 2020, now abandoned.
(60) Provisional application No. 62/800,109, filed on Feb. 1, 2019.

(51) Int. Cl. G16H 20/00; G16H 10/60; G16H 40/67; G06N 20/00; G06N 5/04; H04L 51/02; G16H 50/50

(52) U.S. Cl. CPC G16H 20/00; G16H 10/60; G16H 40/67; G06N 20/00; G06N 5/04; H04L 51/02; G16H 50/50; G16Y 10/60

(57) ABSTRACT

The present disclosure relates to effective, efficient, and economical methods and systems for improving patient engagement, education, and treatment management. In particular, the present disclosure relates to a unique configuration of technology wherein analysis by artificial intelligence may determine improvement to treatment plans and may adjust such treatment plans to improve patient care or reduce cost.

### CROSS REFERENCE TO RELATED APPLICATIONS

This application is a continuation of U.S. patent application Ser. No. 17/454,483, filed on Nov. 10, 2021, which is a continuation of U.S. patent application Ser. No. 17/336,294, filed on Jun. 1, 2021, which is a continuation of U.S. patent application Ser. No. 16/780,892, filed on Feb. 3, 2020, which claims the benefit of and priority to U.S. Provisional Patent Application Ser. No. 62/00,109, filed on Feb. 1, 2019, the entireties of which are hereby incorporated herein by reference.

### BACKGROUND

### Field of the Invention

The present disclosure relates to methods and systems for improving patient engagement, education, and treatment management.

### Description of the Related Art

Overactive bladder (“OAB”) is a chronic disease affecting up to 23% of the United States population. Prevalence of OAB increases with age and sex. Although OAB occurs among men, women are almost twice as likely experience the condition, and over one-third of women over the age of sixty are affected by OAB. Beyond pelvic health, bladder control problems have been associated with a higher incidence of other significant health problems, such as obesity, diabetes, falls, inactivity, depression, and social isolationism.

The first-line treatment, for OAB is behavioral therapy, including education about proper toileting behavior, timed voiding, and training of pelvic floor muscles, typically delivered by a nurse practitioner or pelvic floor physical therapy. If this does not suffice, anticholinergic or other medications are given, followed by minimally invasive neuromodulation, Botox, and finally, sacral neuromodulation surgery. Documentation, adherence, and feedback to the provider are often important with respect to the success of these treatments.

For example, a standard patient tool for OAB diagnosis and treatment is a bladder diary. These require patients to input fluid consumption and output while tracking OAB symptoms as they occur. Although an important tool for data gathering, most bladder diaries are in a paper format, which makes them time-consuming for physicians and patients. Physicians must spend significant time scrutinizing handwriting and making calculations, which then must be entered into databases for analysis. The data gathered from these paper diaries is not always accurate, as patients are often non-compliant because of the time and the organization the diaries require. This typically causes patients to fall behind on their entries, negating the benefits of such bladder diaries entirely.

In addition, many obstacles impair patients from receiving OAB education and treatment, such as medical costs, insurance coverage, and societal stigmas and taboos surrounding bladder issues. Lack of diagnosis and treatment of OAB can also negatively affect a patient's mental state, leading to anxiety and depression that can itself become an additional barrier to effective OAB education and treatment.

Accordingly, there is a need for improved treatments and technologies that provide better and less expensive OAB patient engagement, education, and treatment management.

### SUMMARY

The present disclosure is related to effective, efficient, and economical methods and systems for improving patient engagement, education, and treatment management. In particular, the present disclosure relates to a unique configuration of technology wherein one or more servers may be coupled to a network to provide: a user database containing patient profiles, wherein each patient profile may be capable of storing one or more treatment plans; a content module that may generate the one or more treatment plans, wherein a content map may be also generated as part of each treatment plan; a chatbot module that may utilize the content map to record patient success with respect to the one or more treatment plans; an IoT Module that may obtain IoT data from IoT devices coupled to the one or more servers via the network; and an AI Analytic Module that may evaluate data obtained from one or more patient profiles and the IoT data to determine if modifications would likely improve the success of one or more treatment plans and a such may implement such modifications to the one or more treatment plans if such modifications are determined to likely improve the success of the one or more treatment plans.

### BRIEF DESCRIPTION OF THE DRAWINGS

FIG. 1 illustrates an example of a System for health care management.

FIG. 2 illustrates an example of a System for health care management.

FIG. 3 illustrates an example of a Patient Profile that may be stored in a User Database.

FIG. 4 illustrates an example of a Treatment Plan.

FIG. 5 illustrates a method for administering health care management.

### DETAILED DESCRIPTION OF THE ILLUSTRATED EMBODIMENTS

In the following description, for the purposes of explanation, specific details are set forth in order to provide a thorough understanding of embodiments of the invention. However, it will be apparent that various embodiments may be practiced without these specific details. The figures and description are not intended to be restrictive.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the invention as set forth in the appended claims.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments. As another example, while embodiments are disclosed herein with respect to OAB, other embodiments may address other medical issues such as bowel disorders, eating disorders, pain management, addiction counseling, etc.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

The term “machine-readable storage medium” or “computer-readable storage medium” includes, but is not limited to, portable or non-portable storage devices, optical storage devices, and various other mediums capable of storing, containing, or carrying instruction(s) and/or data. A machine-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-program product may include code and/or machine-executable instructions that may represent a procedure, a function, a subprogram, a program, a routine, a subroutine a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments may be implemented by hardware, software, firmware, middleware, microcode, hardware description languages, or any combination thereof. When implemented in software, firmware, middleware or microcode, the program code or code segments to perform the necessary tasks (e.g., computer-program product) may be stored in a machine-readable medium. A processor(s) may perform the necessary tasks.

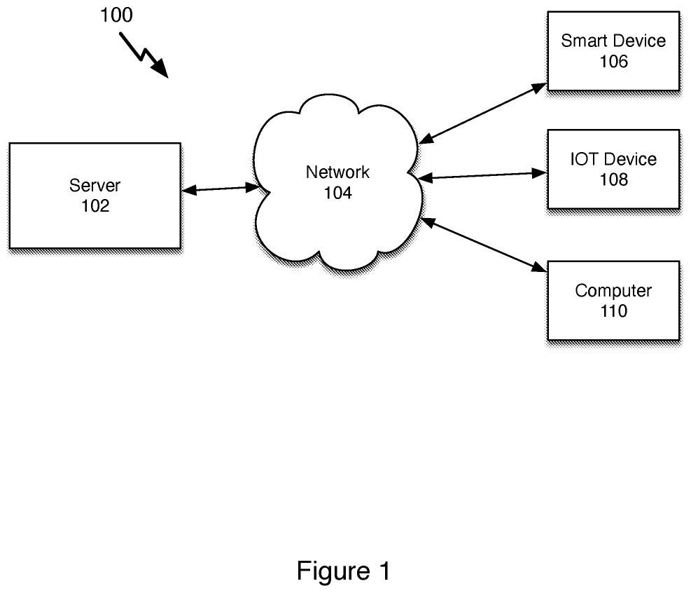

With respect to FIG. 1, an example of a System 100 for health care management is shown. In this embodiment, System 100 may consist of a Server 102 that is connected to a Network 104. Network 104 may be further connected to Smart Device 106, IoT Device 108, and Personal Computer 110.

Server 102 may be comprised of a stand-alone server, multiple or distributed servers, a cloud platform, or the like. Network 104 may include a wireless network, a wired network, a combination of a wired and wireless network, or a network of networks, such as the Internet. A wireless network may include any wireless interface or combination of wireless interfaces (e.g., Zigbee™, Bluetooth™, WiFi™, IR, UWB, WiFi-Direct, BLE, cellular, Long-Term Evolution (LTE), WiMax™, or the like). A wired network may include any wired interface (e.g., fiber, ethernet, powerline ethernet, ethernet over coaxial cable, distal signal line (DSL), or the like). The wired or wireless networks may be implemented using various routers, access points, bridges, gateways, or the like, to connect devices in the local area network 102. Smart Devices 106 may include tablet computers or smart phones such as the iPad™ and iPhone™ from Apple™, Amazon Kindle™, Android™ devices, and so on. IoT Devices 108 may be comprised of computing devices that include sensing control functionality as well as a WiFi™ transceiver radio or interface, a Bluetooth™ transceiver radio or interface, a Zigbee™ transceiver radio or interface, an Ultra-Wideband (UWB) transceiver radio or interface, a WiFi-Direct transceiver radio or interface, a Bluetooth™ Low Energy (BLE) transceiver radio or interface, or any other wireless network transceiver radio or interface that allows the IoT device to communicate with a wide area network and with one or more other devices. In some embodiments, an IoT device does not include a cellular network, transceiver radio or interface, and thus may not be configured to directly communicate with a cellular network. In some embodiments, an IoT device may include a cellular transceiver radio, and may be configured to communicate with a cellular network using the cellular network transceiver radio. Personal Computers 110 may include desktop or laptop computers running MacOS™, Windows™, and so on.

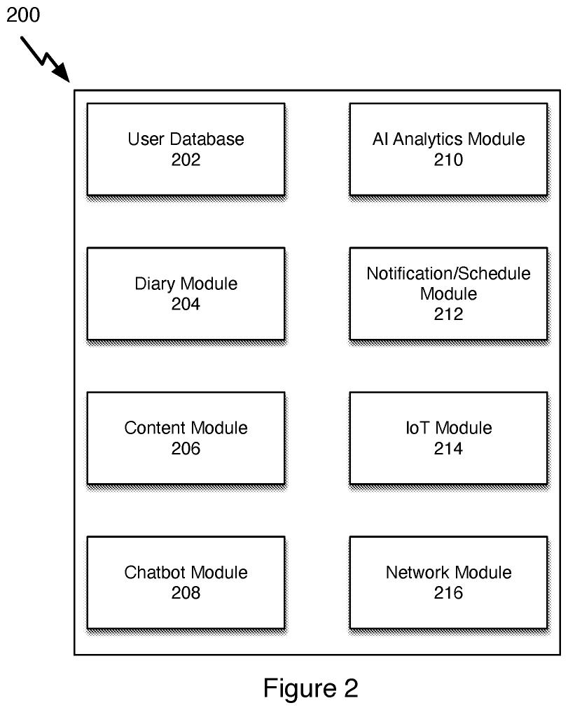

With respect to FIG. 2, an example of a System 200 for health care management that may operate using System 100 is shown. System 200 may include a User Database 202, a Diary Module 204, a Content Module 206, a Chatbot Module 208, an AI Analytic Module 210, a Notification/Schedule Module 212, an IoT Module 214, and a Network Module 216.

User Database 202 may store records of users of System 200, such as patients and providers. Diary Module 204 may facilitate the keeping and processing of diaries associated with users of System 200, such as bladder diaries for OAB patients. Content Module 206 may facilitate the storage, generation, and updating of content for users of System 200, such as educational material, medical protocols, and decision trees. Chatbot Module 208 may provide System 200 with chatbot functionality. AI Analytic Module 210 may facilitate the storage and application of adaptive algorithms to analyze data within System 200. Notification/Schedule Module 212 may facilitate the sending or receiving of notifications or scheduling of events by System 200. IoT Module 214 may facilitate the handling of interactions of System 200 with IoT Devices 108 and the storage of data provided by IoT Devices 108. Network Module 216 may facilitate the communication of System 200 via a network, such as to connect Smart Devices 106, IoT Devices 108, and Personal Computers 110.

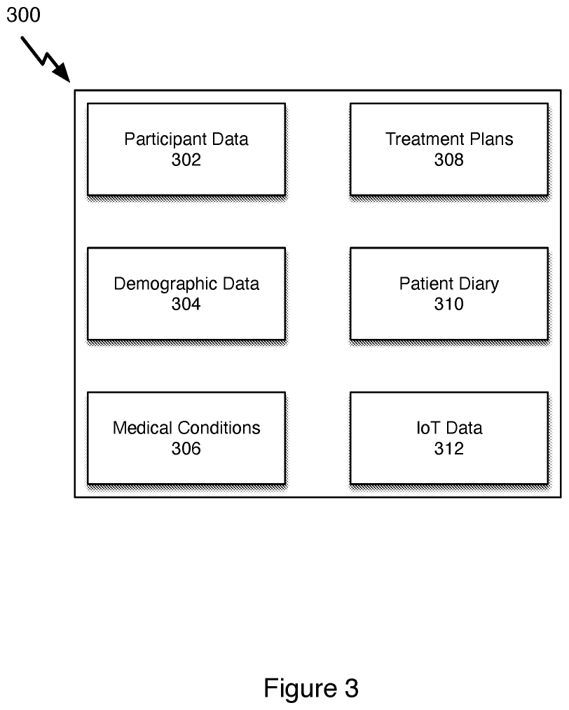

With respect to FIG. 3, an example of a Patient Profile 300 that may be stored in User Database 202 is shown. Participant Data 302 of Patient Profile 300 may contain information identifying the user of the system, such as a patient name, social security, date of birth, place of birth, place of residence, and so on. Participant Data 302 may also include medical and insurance records associated with the user, such as a primary physician, an insurance provider/number, medical imaging records, and so on. In some embodiments, Participant Data 302 may contain information about patient preferences relevant to programming IoT Devices 108.

Demographic Data 304 of Patient Profile 300 may contain information describing the demographic representation of the user, such as sex, age group, race, socioeconomic status, religious affiliation, and so on.

Medication Conditions 306 of Patient Profile 300 may contain information describing pre-existing medication conditions (e.g., diabetes), dietary restrictions (e.g., vegan), allergies, or other quantifiable factors that may be relevant to the selection of medical treatment.

Treatment Plans 308 of Patient Profile 300 may contain treatment plans generated via Content Module 206.

Patient Diary 310 of Patient Profile 300 may contain records entered by a user or generated by System 200 relating to events associated with the user. For example, records in Patient Diary 310 may document liquid intake, toilet visits, urinary discharge volume, urinary discharge intensity, urinary discharge length, patient weight, patient adherence, patient mood, and so on.

Data 312 of Patient Profile 300 may contain information identifying IoT Devices 108 associated with a user. For example, IoT Data 312 may identify that a patient has an Apple™ watch, a smart bathroom scale, a smart beverage 161 device, a smart IoT toilet device, and so on. Information in IoT Data 312 may then be used to facilitate interactions within System 200 between IoT Devices 108 and IoT Module 214. In some embodiments, IoT Data 312 may also store information received by System 200 from to Devices 108 associated with a user. In further embodiments, a user may disable the storage of data received from IoT Devices 108 or selectively delete portions of such data.

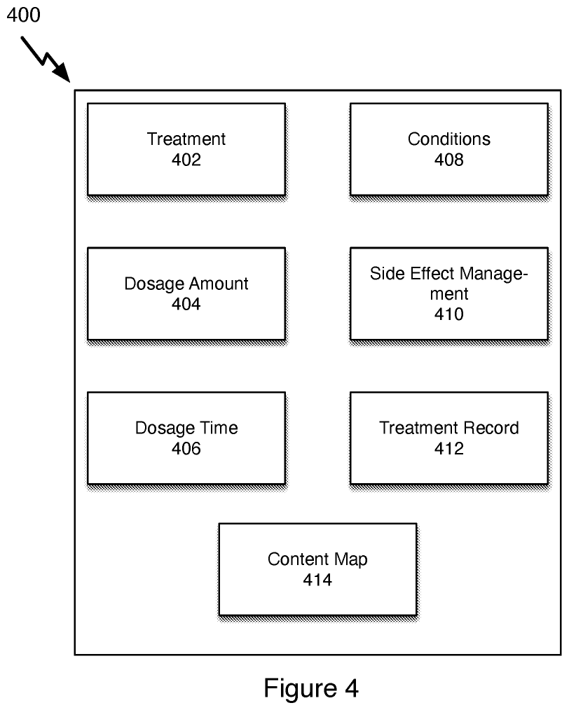

With respect to FIG. 4, an example of a Treatment Plan 400 is shown. Treatment 402 of Treatment Plan 400 may identify the type of treatment, such as a particular therapy that ma involve medication, physical therapy, behavioral modification, and so on, Dosage Amount 404 of Treatment Plan 400 may specify the dosages of any medications associated with the treatment identified in Treatment 402. Dosage Time 406 of Treatment Plan 400 may specify the time or intervals for the consumption or application of any medications associated with the treatment identified in Treatment 402. Conditions 408 of Treatment Plan 400 may specify conditions to be observed when taking any medications associated with, the treatment identified in Treatment 402 (e.g., with meals, avoid alcohol, no driving), Side Effect Management 410 of Treatment Plan 400 may specify potential side effects arising from the treatment identified in Treatment 402 and may also include instructions relating to potential side effects (e.g., reduce dosage, contact doctor, cease medication). Treatment Record 412 of Treatment Plan 400 may contain information recording the effectiveness of the treatment identified in Treatment 402. For example, for each day or step of a treatment plan, the effectiveness of the treatment may be recorded in Treatment Record 412. Effectiveness may be recorded in a variety formats, such as Effective, Partially Effective, or Not Effective. In some embodiments, effectiveness may be rated on a numerical scale (e.g., 1 to 10) or by other metrics. In addition, the value of educational tips in complying with the treatment plan may also be recorded (e.g., helpful, not helpful). Content Map 414 of Treatment Plan 400 may include content for use with the treatment identified in Treatment 402, such as conversation trees, images, illustrations, videos, animations, assessments (e.g., to determine the effectiveness of therapy), interactive tools, and so on.

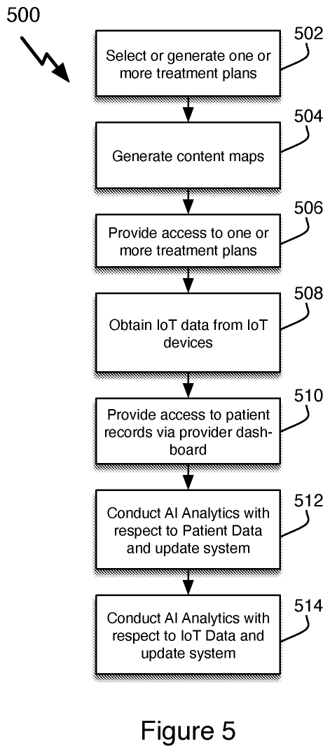

With respect to FIG. 5, a method 500 for administering a health care management is shown. At step 502, one or more treatment plans may be devised by a physician or automatically generated by Content Module 206. Treatment plans may be based upon practice guidelines, public or proprietary care pathways, custom templates, and so on. For example, treatments plans may be devised for OAB from Urogynecology and Urology Practice Guidelines and Renalis™ proprietary care pathways. In some embodiments, treatment plans may allow for adjustment of the therapy by the AI Analytics Module 210. For example, dosage amount and dosage time may be designed as adjustable, such that the AI Analytics Module 210 may optimize these parameters based on analysis of Patient Diary Module 204 or other data. For example, if leakage events occur while sleeping, the AI Analytics Module 210 may shift a dosage time (as recorded in the patient's Treatment Plan 400) closer to the patient's bedtime.

At step 504, content maps may be generated by Content Module 206. Content maps may include any conversation trees, images, illustrations, videos, and animations, assessments, and other interactive tools that may aid in a treatment plan. In some embodiments, content maps may allow for adjustment by the AI Analytics Module 210. For example, based on Treatment Record 412 from multiple patients, AI Analytics Module 210 may determine that certain tips, interactive tools, conversation trees, and so on are more effective or helpful than others. In such an embodiment, AI Analytics Module 210 may then adjust the priority or placement of such allowable elements in the content map so as to present such elements earlier to patients. In another example, AI Analytics Module 210 may determine that certain elements of the content map are more effective for some patients than others based on demographic analysis of patients and their treatment records. In such situations, AI Analytics Module 210 may then adjust the priority or placement of such allowable elements in the content map to suit the demographics of individual patients.

At step 506, a patient user may access one or more treatment plans via a Smart Device 106 or a Personal Computer 110. The patient user may then interact with the content map according to the treatment plan, such as interacting with conversational trees. Conversational trees can be used to educate patients about the treatment plan, to obtain data about adherence to the treatment plan, to obtain the mood of the patient, to obtain data regarding the effectiveness of the treatment plait, to detect events requiring intervention by physicians or other support staff, and so on. The patient may also record entries in Patient Diary Module 204. For example, a patient may note that she urinated at 9 pm and did not encounter any leakage prior to urination.

At step 508, Module 214 may obtain IoT data from IoT Devices 108. In some embodiments, such IoT data may be recorded only on an app on a Smart Device 108 associated with a user. In other embodiments, such IoT data may be recorded in IoT Data 312 associated with a user. IoT Module 214 may also perform analysis of IoT data and accordingly update various parts of the Patient Profile 300 associated with a user.

For example, a patient may have a bathroom scale that updates IoT Module 214 with the patient's weight with each use. IoT Module 214 may then update the Patient Diary 310 associated with the user with a record reflecting the patient's weight at a specific date and time.

As another example, a patient may have a beverage dispenser that has a tilt/movement sensor, programmable buttons, and a measurement component. In some embodiments, IoT Module 214 may rely on Participant Data 302 associated with the patient to program the programmable buttons (e.g., button 1: water, button 2: coffee, button 3: juice). The measurement component may be a float within an interior groove of the beverage dispenser whose vertical height is determined by an electronic sensor strip within or adjacent to the groove, in some embodiments, the electronic sensor strip may determine the location of the float via an array of optical sensors (e.g., the float blocks a reflective or lighted strip on the other side of the groove) or an array of sensors able to detect a capacitive or magnetic aspect of the float. Accordingly, when the tilt/movement sensor is triggered the beverage dispenser may determine that a possible consumption of liquid has occurred. Once the beverage, dispenser has determined that it is at rest again based on the tilt/movement sensor, it may then use the measurement component to determine if a change in liquid height has occurred. If so, the beverage dispenser may calculate the volume of liquid consumed based on geometric equations (e.g., volume=π*r²*h, where r is the radius of the beverage container and h is the change in height) and may then update IoT Module 214 with the time at which consumption occurred and volume of liquid consumed. If the patient has used the programmable buttons to select a beverage type, then the beverage container may also include that information with an update. In some embodiments, the beverage container may also reset the beverage type when it determines that the liquid has been consumed, such as by information obtained from the measurement component or the tilt/movement sensor. In further embodiments, the beverage container may also detect when liquid has been added such as by information obtained from the measurement component or the tilt/movement sensor, and may thus present or send a request for a beverage type (e.g., lights on the programmable buttons may flash, a phone or watch displays a notification to select the beverage type). In alternative embodiments, the beverage type may be selected on an app on a phone or watch, which then updates the beverage sensor or IoT Module 214. In view of the above embodiments, to Module 214 may update the Patient Diary Module 204 via information received from the beverage dispenser.

As another example, a patient may have a smart toilet device that has a measurement component. For example, the smart toilet device may have an optical camera that can read graduated markings on the side of a toilet bowel. In some embodiments, the graduated markings may be etched into the bowl. In other embodiments, the graduated markings may be placed in the howl with an adhesive label. Alternatively, the smart toilet device may use a laser to determine the height of the water based on analysis of the reflection of the laser from the surface. In such embodiments, a user may be instructed to pour pre-determined amounts of water (e.g., 100 mL, 200 mL) via an app or System 200 to associate measurements in height with a specific volume of water in the toilet. Based on such methods, the smart toilet device may update IoT Module 214 with the volume of discharge.

In some embodiments, the smart toilet device may have a microphone. The smart toilet device may then be triggered (e.g., by a button or load sensor) to record audio of urination. The smart toilet device may then update IoT Module 214 with audio data. IoT Module 214 or AI Analytics Module 210 may then analyze the audio to determine when urination began and ended, thereby determining the length of urination and any pauses. In addition, the audio may be analyzed based on pitch or other audio elements to determine an intensity of urination or other characteristics of urination. In further embodiments, based on the length of urination and intensity of urination derived from audio data, IoT Module 214 or AI Analytics Module 210 may determine an estimated volume of discharge. In some embodiments, the smart toilet device may also have an audio element to broadcast an audiotraining signal, such as a chirp, to assist the IoT Module 214 or AI Analytics Module 210 to evaluate acoustic properties of the discharge environment, which may then be used to improve acoustic analysis of the urinary discharge event.

In some embodiments, the smart toilet sensor may be a keychain device (e.g., a microphone and button on a stick) that a user can point into the toilet. In some embodiments, the processing of audio data described above by IoT Module 214 or AI Analytics Module 210 may be done with an app on the patient's Smart Device 106, such that only data derived from the audio processing is transmitted to IoT Module 214.

As another example, a patient may be wearing a smart watch that may update IoT Module 214 with information regarding the patient's activity level, sleep patterns, location and so on. If the smart watch is configured to provide IoT Module 214 with such information, then IoT Module 214 may then update Patient Profile 300 with any portion of that information which Treatment Plan 400 indicates is relevant.

At step 510, a physician may access data within a Patient Profile 300 of a patient associated with the physician via a Smart Device 106 or a Personal Computer 110. For example, a physician may use a provider dashboard to review symptoms, treatment adherence, treatment effectiveness, patient mood history, and so on with respect to a Patient Profile 300 of a patient associated with the physician. In some embodiments, the provider dashboard may also provide a communication channel between the participant and the physician. For example, the provider dashboard may allow for the physician to send notifications to the patient via Notification/Schedule Module 212. As another example, the provider dashboard may also allow the physician to schedule appointments with the patient via Notification/Schedule Module 212. In some embodiments, information contained in a patient's Treatment Plan 400 may result in notifications or scheduling requests being sent via Notification/Schedule Module 212 to the provider dashboard. In various embodiments, the provider dashboard may be provided as a module on Server 102, as a module on Personal Computer 110, or both. In some embodiments, provider dashboard may also be used by medical staff other than physicians, such as physical therapists, counselors, and so on.

Provider dashboard may also allow a physician to review modifications made by AI Analytics Module 210 to a patient's Treatment Plan 400 and may further allow the physician to disable or enable such modifications. For example, if AI Analytics Module 210 modified a patient's Treatment Plan 400 to provide for a lower dosage amount doe to age, the physician may disable the modification, Alternatively, allowable changes to a Treatment Plan 400 by AI Analytics Module 210 in some instances may require physician approval in order to be implemented. In such instances, the physician may review the suggested modification provided by AI Analytics Module 210, which may include AI Analytics Module 210 providing justification for such a modification, and then the physician may choose to enable or ignore the suggested modification. For example, if AI Analytics Module 210 recommends a change in dosage amount, it may also provide that the justification for the modification, such as that patients of a similar demographic statistically have a higher rate of effectiveness at the recommended change in dosage amount. However, the physician may be aware of other relevant factors not considered by AI Analytics Module 210 and thus may decide to not enable the modification. Accordingly, with respect to discussion herein regarding AI Analytics Module 210 making modifications, it should be understood that such modifications may be subject to such a review and approval process in various embodiments.

At step 512, AI Analytics Module 210 may review Patient Profiles 300 individually or in aggregate to statistically evaluate any outcomes (e.g., effectiveness, mood, patient understanding of education material) within Treatment Plans 400 of Patient Profiles 300. For example, AI Analytics Module 210 may look to see if any outcomes are statistically dependent on demographic factors, if any differences in outcomes between patients suggest that elements of a treatment plan should be adjusted to improve effectiveness, or, if adjusting any aspects of a treatment plan in order to reduce costs results in an undesirable outcome (e.g., decreased effectiveness, decreased mood, impaired patient understanding).

At step 514, AI Analytics Module 210 may review Patient Profiles 300 individual or in aggregate to statistically evaluate IoT Data 312, which may also include IoT data from other sources such as from IoT Module 214, Smart Devices 106, IoT Devices 110, and so on. In some embodiments, such analysis may also include other data farm Treatment Plans 400 of Patient Profiles 300. For example, AI Analytics Module 210 may detect that certain events recorded by IoT data (e.g., irregular sleep patterns, weight fluctuations) may adversely affect outcomes in Treatment Plans 400 of Patient Profiles 300. AI Analytics Module 210 may then store such detected adverse patterns and may also notify patients, physicians, or both via Notification/Schedule Module 212 that one or more detected adverse patterns may be affecting Treatment Plans 400 associated with the patients. For example, AI Analytics Module 210 may generate a message describing the nature of detected adverse pattern (e.g., irregular sleep patterns) and the impact it may be having on patient's treatment plan (e.g., reduced effectiveness). After which AI Analytics Module 210 may send, the message to the patient via Notification/Schedule Module 212. In addition, AI Analytics Module 210 may receive feedback from providers on detected adverse patterns via Notification/Schedule Module 212 that indicates that the detected adverse pattern is valid or invalid. For example, if a detected adverse pattern is, found by providers to arise from faulty or unreliable IoT data or other patient data, a provider may indicate that the detected adverse pattern is invalid. For example, if a subset of patients is entering false data that contradicts IoT data, this may result in invalid adverse data patterns. In addition, IoT devices may have defects that are later found to make their measurements unreliable.

In some embodiments, a Treatment Plan 400 may allow for adjustments based on adverse data patterns found by AI Analytics Module 210. For example, if AI Analytics Module 210 determines that intense physical activity requires a change in the treatment plan (e.g., more frequent toilet visits), AI Analytics Module 210 may accordingly adjust the treatment plan if so allowed. As another example, AI Analytics Module 210 may find an adverse data pattern based on demographic patterns, such as educational impairment or change in patient mood, that may be corrected by changes to a conversational tree. For example, educational material present in a conversation tree may have multiple levels of sophistication in its presentation of a topic (e.g., educational material for teenagers, educational material for adults). In such an instance, AI Analytics Module 210 may determine based on demographic factors an adverse data pattern, such as that ESL adult patients have better outcomes with educational material for teenagers than with educational material for adults. AI Analytics Module 210 may then accordingly adjust treatment plans for ESL adult patients if so allowed to use educational material for teenagers instead of educational material for adults.

In general, AI Analytics Module 210 may statistically evaluate data within System 200 to the extent it has permission to do so to identify factors with respect to treatment plans that affect compliance, symptom management, effects of educational material, effects of behavioral change strategies, recorded emotional or physical states of patients, and so on. AI Analytics Module 210 may then provide notify patients, providers, or both as to the presence of such factors if relevant to the patient or the patient's treatment plan. In some embodiments, the identification of such factors may result in modification by AI Analytics Module 210 of Treatment Plans 400, which may be subject to physician review and approval. In further embodiments, the identification of such factors may result in modification by AI Analytics Module 210 of content maps generated by Content Module 206, which may be subject to physician review and approval. For example, AI Analytics Module 210 may determine that with respect to a default content map generated by Content Module 206 that a modification of 60% is required based on the existing patient pool using the default content map, but that if the content map was switched to a flew default that a modification of only 30% would be required. In such instances, such a modification may be automatically permitted, such as where the change merely minimizes the consumption of resources by System 200. In other instances, such a modification may require review and approval, such as by one or more physicians or a system provider of System 200.

For purposes of illustration, an example is provided as follows tor a patient who suffers from OAB and is managed by System 200. The OAB patient may make an appoint with a urology practice, during which consultation the OAB patient is requested to download an application to the OAB's smart device to interact with System 200. Once the OAB patient downloads the application, the OAB patient may enter an authentication code associating the OAB patient with a physician, request access via a physician from a physician directory, or so on to establish a patient user account with System 200. OAB patient may then enter personal and medical information upon obtaining an account, which is then entered into a Patient Profile 300 for the OAB patient. The physician may select a therapy or diagnosis for the OAB Patient. This may then result in Content Module 206 generating a treatment plan based on selected therapy or diagnosis, which may also include generating a content map as part of the treatment plan. The Content Module 206 may then update the Patient Profile for the OAB patient with the treatment plan and content map if applicable. The treatment plan may education the OAB user regarding the therapy and then may instruct the OAB user to document urination/voiding events for 72 hours. Accordingly, the OAB patient may document such events in System 200 via Smart Device 106, where it may be stored in the Patient Diary 310. In addition, OAB patient may interact with System 200 via Chatbot Module 208 to help determine goals (e.g., I want to go two hours without leaking; I want to stop wearing pads), patient mood, and so on. During the initial 72 hours, the treatment plan may also instruct the OAB patient to document liquid consumption. The OAB patient may perform this using a Smart Device 106 connected to System 200, a smart beverage container as described above connected to System 200, a combination of both, or other methods. In addition, the OAB patient may also be requested to provide an indication of the, strength of the urge to urinate, which the OAB patient may provide via Smart Device 106 or IoT Device 110. In some embodiments, AI Analytics Module 210 may detect based on the original 72 hours of data when urination is likely to occur and send a notification asking if the OAB patient has experienced a recent urination event. If the OAB patient is providing geolocation data to System 200, such as by a Smart Device 106 or a smart watch, AI Analytics Module 210 may observe that OAB patient is close to an area associated with urination events and thus AI Analytics Module 210 may send a notification asking if the OAB patient has experienced a recent urination event. Given the baseline data obtained by System 200 during the first 72 hours, AI Analytics Module 210 may calculate total volume intake per day, total volume output per day, total number of voids per day, average interval between voids, and so on. Based on such analysis, AI Analytics Module 210 may then further determine a diagnosis, such as that OAB patient has an overactive bladder, stress incontinence, mixed incontinence, urinary retention, and so on. AI Analytics Module 210 may then augment the treatment plan, which may be subject to the approval of the physician, with additional treatment plans generated via Content Module 206 in response to the diagnosis. As the DAB patient proceeds with the one or more treatment plans, System 200 may provide positive reinforcement and behavioral coaching, in order to improve the OAB patient's success and mood with respect to the treatment plans, which may be adjusted by AI Analytics Module 210 based on the DAB patient's progress with the one or more treatment plans as measured by effectiveness, adherence, mood and so on. If the OAB patient is determined to have not satisfied selected criteria (e.g., due to a lack of adherence to a treatment plan), System 200 may document the issue and forward such documentation to the physician. As the OAB patient interacts with System 200, AI Analytics Module 210 may evaluate the success of treatment plans based on metrics such as adherence, decline in physician visits, changes in mood or effectiveness, reduction in care costs, and so on. Further, as AI Analytics Module 210 obtains more data in this regard from the OAB patient or other similar patients, it may then individually modify (or in general) the treatment plans to improve the likelihood of the success by the OAB patient. In this manner, once the initial 72 hour period has passed, the OAB patient in this example will not only be able to continue documenting liquid consumption and urination data as before, but also will be able to engage with adaptive treatment plans that are dynamically tailored to the OAB patient by the AI Analytics Module 210. Accordingly, such analysis, feedback, and modification by the AI Analytics Module 210 may potentially achieve better health outcomes more rapidly for the OAB patient, in addition to potentially achieving other benefits described herein, such as a lower overall cost of treatment.

Substantial variations may be made in accordance with specific requirements. For example, customized hardware might also he used, and/or particular elements might be implemented in hardware, software (including portable software, such as applets, etc.), or both. Further, connection to other access or computing devices such as network input/output devices may be employed.

In the foregoing specification, aspects of the invention are described with reference to specific embodiments thereof, but those skilled in the art will recognize that the invention is not limited thereto. Various features and aspects of the above-described invention may be used individually or jointly. Further, embodiments can be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate embodiments, the methods may be performed in a different order than that described. It should also be appreciated, that the methods described above may be performed by hardware components or may be embodied in sequences of machine-executable instructions, which may he used to cause a machine, such as a general-purpose or special-purpose processor or logic circuits programmed with the instructions to perform the methods. These machine-executable instructions may he stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

While embodiments of the application have been described in detail herein, it is to be understood that the inventive concepts may be otherwise variously embodied and employed, and that the appended claims are intended to be construed to include such variations, except as limited by the prior art.

What is claimed is:

1. A system for improving patient engagement, education, and treatment management comprising;
one or more processors, and
at least one non-transitory computer-readable storage medium having stored therein instructions which, when executed by the one or more processors, cause the system to:
provide a user database containing patient profiles, wherein each patient profile is capable of storing one or more treatment plans;
generate a content module that contains the one or more treatment plans, wherein a content map containing one or more conversation trees, images, illustrations, videos or animations is also generated as part of each treatment plan;
utilize the content map to record patient success with respect to the one or more treatment plans;
obtain IoT data from IoT devices coupled to the one or more processors via a network; and
evaluate data obtained from one or more patient profiles and the IoT data to determine if modifications to the content map would likely improve the success of one or more treatment plans and implement such modifications to the content map if such modifications are determined to likely improve the success of the one or more treatment plans.

2. The system of claim 1, wherein the modifications to the content map include adjusting the priority or placement of elements in the content map so as to present such elements to a patient in a different order.

3. The system of claim 1, wherein the modifications to the content map include adjusting the priority or placement of elements in the content map with respect to patient demographics.

4. The system of claim 2, wherein the at least one non-transitory computer-readable storage medium further stores instructions which, when executed by the one or more processors, cause the system to:
evaluate patient responses to the one or more conversational trees in the content map to determine patient adherence to the one or more treatment plans, the mood of the patient, or the need for medical intervention.

5. The system of claim 1, wherein the at least one non-transitory computer-readable storage medium further stores instructions which, when executed by the one or more processors, cause the system to:
process audio or video in the IoT data to estimate the volume of a urinary discharge.

6. The system of claim 5, wherein the at least one non-transitory computer-readable storage medium further stores instructions which, when executed by the one or more processors, cause the system to:
process audio or video in the IoT data to estimate the intensity or length of the urinary discharge.

7. The system of claim 1, wherein the system further includes a beverage dispenser that has a tilt/movement sensor and a measurement component and is configured to provide beverage dispenser IoT data regarding when consumption of a liquid has occurred or the volume of the liquid consumed.

8. The system of claim 7, wherein the beverage dispenser has programmable buttons for specifying one or more liquid types and wherein the beverage dispenser IoT data is capable of including a selection of the one or more liquid types.

9. The system of claim 4, wherein the at least one non-transitory computer-readable storage medium further stores instructions which, when executed by the one or more processors, cause the system to:
provide a dashboard interface allowing for the review of the modifications and to allow a user to enable or disable the modifications.

10. The system of claim 9, wherein the at least one non-transitory computer-readable storage medium further stores instructions which, when executed by the one or more processors, cause the system to:
analyze the IoT data across multiple patient profiles to detect adverse data patterns in the IoT data that affect patient outcomes and wherein the dashboard interface allows for the review of the adverse data patterns and to allow a user to select whether individual adverse data patterns are valid or invalid.

11. A computer-operable method for improving patient engagement, education, and treatment management comprising the steps of:
providing a user database containing patient profiles, wherein each patient profile is capable of storing one or more treatment plans;
generating a content module that contains the one or more treatment plans, wherein content map containing one or more conversation trees, images, illustrations, videos, or animations, is also generated as part of each treatment plan;
utilizing the content map to record patient success with respect to the one or more treatment plans;
obtaining IoT data from IoT devices via a network; and
evaluating data obtained from one or more patient profiles and the IoT data to determine if modifications to the content map would likely improve the success of one or more treatment plans and implementing such modifications to the content map if such modifications are determined to likely improve the success of the one or more treatment plans.

12. The computer-operable method of claim 11, wherein the modifications to the content map include adjusting the priority or placement of elements m the content map so to present such elements to a patient in a different order.

13. The computer-operable, method of claim 11, wherein the modifications to the content map include adjusting the priority or placement of elements in the content map with respect to patient demographics.

14. The computer-operable method of claim 12, further comprising the step of:
evaluating patient responses to the one or more conversational trees in the content map to determine patient adherence to the one or more treatment plans, the mood of the patent, or the need for medical intervention,

15. The computer-operable method of claim 11, further comprising the step of processing audio or video in the IoT data to estimate the volume of a urinary discharge,

16. The computer-operable method of claim 15, further comprising the step of:
processing audio or video in the IoT data to estimate the intensity or length of the urinary discharge.

17. The computer-operable method of claim 11, further comprising the step of:
receiving from a beverage dispenser that has a tilt/movement sensor and a measurement component beverage dispenser IoT data regarding when consumption of a liquid has occurred or the volume of the liquid consumed.

18. The computer-operable method of claim 17, wherein the beverage dispenser IoT data is capable of including a selection of the one or more liquid types.

19. The computer-operable method of claim 14, further comprising the step of:
providing a dashboard interface allowing for the review of the modifications and to allow a user to enable or disable the modifications.

20. The computer-operable method of claim 19, further comprising the step of:
analyzing the IoT data across multiple patient profiles to detect adverse data patterns in the IoT data that affect patient outcomes,
wherein the dashboard interface allows for the review of the adverse data patterns and to allow a user to select whether individual adverse data patterns are valid or invalid.
